(19) United States
(12) Patent Application Publication
(10) Pub. No.: US 2025/0066467 A1
(43) Pub. Date: Feb. 27, 2025

(54) METHODS FOR THE TREATMENT OF MYASTHENIA GRAVIS

(71) Applicant: Tourmaline Bio, Inc., New York, NY (US)

(72) Inventors: Sandeep KULKARNI, New York, NY (US); Yung CHYUNG, New York, NY (US); Susan Dana JONES, New York, NY (US); Ryan IARROBINO, New York, NY (US); W. Bradford MIDDLEKAUFF, New York, NY (US)

(21) Appl. No.: 18/721,059

(22) PCT Filed: Nov. 16, 2023
(86) PCT No.: PCT/US2023/080049

§ 371 Date: Jun. 17, 2024

Related U.S. Application Data
(60) Provisional application No. 63/384,865, filed on Nov. 23, 2022.

(51) Int. Cl. C07K 16/24; A61K 39/00

(52) U.S. Cl. CPC C07K 16/248; A61K 2039/545; C07K 2317/565

(57) ABSTRACT

The present disclosure provides methods of treating myasthenia gravis comprising subcutaneously administering to a patient in need thereof a therapeutically effective dose of an anti-interleukin-6 (anti-IL-6) antibody or antibody fragment. Further provided herein are pharmacologically active agents, compositions, methods and/or dosing schedules for the treatment of myasthenia gravis.

### CROSS-REFERENCE TO RELATED APPLICATIONS

This application claims priority to U.S. Provisional Patent Application No. 63/384,865, filed Nov. 23, 2022, which is incorporated by reference herein in its entirety for all purposes.

### DESCRIPTION OF THE TEXT FILE SUBMITTED ELECTRONICALLY

The contents of the electronic listing sequence (TOUR_003_01WO_SeqList_ST26.xml; Size: 14,166 bytes; and Date of Creation: Nov. 9, 2023) are herein incorporated by reference in their entirety.

### TECHNICAL FIELD

The disclosure relates to therapeutic antibody molecules and treatments for myasthenia gravis (MG).

### BACKGROUND

Myasthenia gravis (MG) is an autoimmune neuromuscular disorder characterized by fluctuating motor weakness involving ocular, bulbar, limb, and/or respiratory muscles. The weakness is caused by specific autoantibodies against the acetylcholine receptor (AChR), muscle-specific kinase (MuSK) or other AChR-related proteins in the postsynaptic muscle membrane. MG is the most common disorder of neuromuscular transmission.

The existing standard of care in the management of myasthenia gravis includes ‘broad-spectrum’ immunosuppressive treatment (IST) with medications such as corticosteroids, azathioprine, mycophenolate, methotrexate, cyclosporine, tacrolimus, and immunomodulatory treatments such as plasma exchange (PLEX) and intravenous immunoglobulin (IVIG). While these treatments are time-tested and remain the commonly used agents for MG, the disadvantages are many such as increased susceptibility to life threatening infections, a wide range of deleterious systemic side effects, delayed onset of action and increased long term risk of malignancy and drug toxicity. Thus a significant unmet medical need exists for an effective chronic-intermittent treatment with less toxicity, rapid onset of action with possibly sustained remission and cure, and increased convenience for patients with MG.

### SUMMARY

Provided herein is a method of treating myasthenia gravis (MG) comprising administering to a patient in need thereof a therapeutically effective dose of an anti-interleukin-6 (anti-IL-6) antibody or antibody fragment having the variable heavy (VH) complementarity-determining regions (CDRs) as defined in SEQ ID NOs 2, 3 and 4, and the variable light (VL) CDRs as defined in SEQ ID NOs 8, 9 and 10.

In some embodiments, the anti-IL-6 antibody or antibody fragment comprises a heavy chain polypeptide comprising a polypeptide having at least about 98% identity to SEQ ID NO: 1 and a light chain polypeptide comprising a polypeptide having at least about 98% identity to SEQ ID NO: 7. In one aspect, the anti-IL-6 antibody or antibody fragment comprises a heavy chain polypeptide having the sequence of SEQ ID NO: 1 and a light chain polypeptide having the sequence of SEQ ID NO: 7.

In some embodiments, the anti-IL-6 antibody or antibody fragment containing said CDRs as described herein is contained in a pharmaceutical composition that comprises said anti-IL6 antibody or antibody fragment and a pharmaceutically acceptable carrier.

In some embodiments, the therapeutically effective dose of the present disclosure is between 5 mg to 100 mg. In some embodiments, the therapeutically effective dose is about 5, about 7.5, about 10, about 15, about 20, about 25, about 30, about 35, about 40, about 45, about 50, about 55, about 60, about 65, about 70, about 75, about 80, about 85, about 90, about 95, or about 100 mg of the anti-IL-6 antibody or antibody fragment.

In some embodiments, therapeutically effective dose of the antibody, or an antigen binding fragment thereof may be administered by any suitable route including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, hypospray, intravaginal or rectal routes. In one embodiment, the therapeutically effective dose is administered subcutaneously. In some embodiments, the antibody, or an antigen binding fragment thereof is treated through home administration (e.g. self-administered, or administered by caregiver or by visiting healthcare professional).

In some embodiments, the dosing schedules for the anti-IL-6 antibody or antibody fragment is every 1 week to every 24 weeks. In one embodiment, the therapeutically effective dose is administered every 4, 8, 12 or 24 weeks.

In some embodiments, the treatment may be provided over a total duration of about 1 month, about 2 months, about 3 months, about 4 months, about 5 months, about 6 months, about 7 months, about 8 months, about 9 months, about 10 months, about 11 months, about 12 months, about 14 months, about 16 months, about 18 months, about 20 months, about 22 months or about 24 months.

In some embodiments, the method of the present disclosure comprises: (a) administering a loading dose of the anti-IL-6 antibody or antibody fragment to the patient for at least the first does during a loading regimen; and (b) thereafter administering a maintenance dose of the anti-IL-6 antibody or antibody fragment subcutaneously to the patient during a maintenance regimen. In some embodiments, the loading regimen comprises administering the loading dose every 1 week, every 2 weeks, or every 4 weeks. In some embodiments, the maintenance regimen comprises administering the maintenance dose every 4 weeks, every 8 weeks, every 12 weeks, or every 24 weeks. In some embodiments, the loading dose is greater than or equal to the maintenance dose. In some embodiments, the loading dose is less than the maintenance dose. In some embodiments, the loading dose is between 5 mg to 100 mg. In some embodiments, the maintenance dose is between 5 mg to 100 mg. In one embodiment, the loading regimen comprises one loading dose of 50 mg, and the maintenance regimen comprises the maintenance dose of 20 mg every 4 weeks for a total of 24 weeks. In another embodiment, the loading regimen comprises one loading dose of 20 mg, and the maintenance regimen comprises the maintenance dose of 10 mg every 4 weeks for a total of 24 weeks

In some embodiments, the patient being treated in accordance with methods of the disclosure is positive for anti-Acetylcholine Receptor (AChR), or anti-muscle specific kinase (MuSK) antibody. In some embodiments, the patient has seronegative MG. In some embodiments, the patient has elevated high-sensitivity C-reactive protein (hsCRP) of at least 2 mg/L, 5 mg/L, or 10 mg/L. In some embodiments, the patient has elevated serum IL-6. In some embodiments, the patient has elevated red blood cell distribution width (RDW) of at least 13% or in the highest quartile. In some embodiments, the patient has Myasthenia Gravis Foundation of America (MGFA) class II. In some embodiments, the patient has MGFA class III. In some embodiments, the patient has MGFA class IV. In some embodiments, the patient is naïve to a pharmacologic disease-modifying MG treatment. In one embodiment, the pharmacologic disease-modifying MG treatment comprises acetylcholinesterase inhibitor therapy. In some embodiments, the patient has no or inadequate response to efgartigimod therapy. In some embodiments, the patient has a response to efgartigimod therapy followed by a relapse. In some embodiments, the patient has no or inadequate response to glucocorticoid therapy. In some embodiments, the patient has a response to glucocorticoid therapy followed by a relapse. In some embodiments, the patient has no or inadequate response to intravenous immunoglobulin (IVIG) therapy. In some embodiments, the patient has a response to IVIG therapy followed by a relapse. In some embodiments, the patient has no or inadequate response to plasmapheresis therapy. In some embodiments, the patient has a response to plasmapheresis therapy followed by a relapse. In some embodiments, the patient has no or inadequate response to non-steroidal immunosuppressant therapy. In some embodiments, the patient has a response to non-steroidal immunosuppressant therapy followed by a relapse. In one embodiment, the non-steroidal immunosuppressant therapy comprises mycophenolate mofetil, cyclophosphamide, cyclosporine, azathioprine, methotrexate, and tacrolimus. In some embodiments, the patient has no or inadequate response to thymectomy. In some embodiments, the patient has a response to thymectomy followed by a relapse.

In some embodiments, the method of treatment as described herein achieves one or more of the following results:
(a) a reduction in serum concentrations of anti-AChR antibody from baseline by at least 40%, 45%, or 50%;
(b) a reduction in Myasthenia Gravis Activities of Daily Living (MG-ADL) score from baseline by at least 2, 2.5, 3, 3.5, 4, 4.5 or 5 points;
(c) a reduction in Quantitative Myasthenia Gravis (QMG) score from baseline by at least 3, 3.5, 4, 4.5, or 5 points;
(d) a reduction in Myasthenia Gravis Composite (MGC) score from baseline by at least 3, 3.5, 4, 4.5, or 5 points; or
(e) a reduction in Myasthenia Gravis Quality of Life 15 Scale (MG-QOL 15r) score from baseline by at least 4, 5, 6, 7, 8, or 9 points.

In some embodiments, the probability of the reduction in serum concentrations of anti-AChR antibody is at least 40%, 50%, 60%, 70%, 80%, or 90%. In some embodiments, the probability of the reduction in MG-ADL score is at least 50%, 60%, 70%, 80%, or 90%. In some embodiments, the probability of the reduction in QMG score is at least 50%, 60%, 70%, 80%, or 90%. In some embodiments, the probability of the reduction in MGC score is at least 50%, 60%, 70%, 80%, or 90%.

In some embodiments, the treatment result is achieved within 16 weeks, 12 weeks, 8 weeks, or 4 weeks. In some embodiments, the treatment result is achieved during a long-term treatment. In one embodiment, the long-term is more than 24 weeks, 48 weeks, 72 weeks, or 96 weeks.

In some embodiments, the treatment result is sustained for at least 8 weeks, 12 weeks, 16 weeks, 20 weeks, 24 weeks, 36 weeks, 48 weeks, or 52 weeks after the last dose administration. In some embodiments, the reduction in serum concentrations of anti-AChR antibody by at least 40%, 45%, or 50% from baseline is sustained for at least 12 weeks after the last dose administration.

In some embodiments, the probability of relapse is less than 50%, 40%, 30%, 25%, 20%, 15%, 10%, or 5% after 12 weeks following the last dose administration.

In some embodiments, the incidence of anti-drug antibodies to the anti-IL-6 antibody or antibody fragment is less than 40%, 30%, 25%, 20%, 15%, 10%, or 5%.

In some embodiments, the methods described herein further comprise a step of treating a subject with an additional form of therapy. In some embodiments, the additional form of therapy comprises administering one or more therapeutic agent in addition to the anti-IL-6 antibody or antibody fragment as described herein. The therapeutic agents include, but are not limited to, a second antibody (e.g., an anti-IL-1 antibody, anti-IGF-1 receptor antibody, anti-VEGF antibody, and/or anti-IL17a antibody), a soluble receptor (e.g., soluble IL-1 receptor, soluble TNF-alpha receptor), an anti-inflammatory agent (e.g., paclitaxel, docetaxel, cisplatin, doxorubicin, prednisone, mitomycin, progesterone, tamoxifen, or fluorouracil), or other common myasthenia gravis medication (e.g., acetylcholinesterase inhibitors; glucocorticoids or nonsteroidal immunosuppressive and immunomodulatory agents; or therapeutic plasma exchange or intravenous immune globulin).

In some embodiments, provided are pharmacologically active agents, compositions, methods and/or dosing schedules that have certain advantages compared to the agents, compositions, methods and/or dosing schedules that are currently used and/or known in the art, including the ability to dose less frequently or to administer lower doses to obtain equivalent effects in inhibiting IL-6 mediated signaling.

### BRIEF DESCRIPTION OF THE DRAWINGS

FIG. 1 presents the schematic for a Phase 2 randomized, double-masked, placebo-controlled, dose-ranging study of TOUR006 with treatment extension in patients with myasthenia gravis.

FIG. 2A and FIG. 2B illustrate the predicted percentage changes of CRP over the treatment period for population A (FIG. 2A) and population B (FIG. 2B), respectively, under the dose regimen of 50 mg LD, followed by 20 mg Q4W starting at 4 weeks.

FIG. 3A and FIG. 3B illustrate the predicted percentage changes of CRP over the treatment period for population A (FIG. 3A) and population B (FIG. 3B), respectively, under the dose regimen of 20 mg LD, followed by 10 mg Q4W starting at 4 weeks.

### DETAILED DESCRIPTION

Provided herein are methods of treating myasthenia gravis (MG) comprising subcutaneously administering to a patient in need thereof a therapeutically effective dose of an anti-interleukin-6 (anti-IL-6) antibody or antibody fragment.

Further provided herein are pharmacologically active agents, compositions, methods and/or dosing schedules for the treatment of MG.

### Antibodies

Provided herein are antibodies and antigen-binding fragments thereof that specifically bind IL-6. Antibodies and antigen-binding fragments disclosed herein specifically bind human IL-6. In some embodiments, an antibody may be specific for only human IL-6 and may exhibit no non-human cross-reactivity.

As used herein, the term “antibody” refers to immunoglobulin (Ig) molecules and immunologically active portions or fragments of immunoglobulin molecules, i.e., molecules that contain an antigen-binding site that specifically binds (immunoreacts with) an antigen (e.g., IL-6). By “specifically binds” or “immunoreacts with” is meant that the antibody reacts with one or more antigenic determinants of the desired antigen and does not react with other polypeptides. In some embodiments, an antibody is said to specifically bind an antigen when it preferentially recognizes its target antigen in a complex mixture of proteins and/or macromolecules. In some embodiments, an antibody “specifically binds” IL-6 if the antibody binds IL-6 with greater affinity, greater avidity, more readily and/or for greater duration than it binds other polypeptides.

The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. Each light chain comprises a light chain variable domain (abbreviated herein as VL domain) and a light chain constant region. The light chain constant region comprises one domain, CL. The VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU numbering system. The Fc region of an immunoglobulin generally comprises two constant domains, CH2 and CH3.An Fc region can be present in dimer or monomeric form. The Fc region binds to various cell receptors, such as Fc receptors, and other immune molecules, such as complement proteins.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH and a VL. Generally IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

The term “antigen-binding portion” or “antigen-binding fragment” of an antibody (or “antibody portion” or “antibody fragment”), as used herein, refers to one or more fragments of an antibody that retain the ability to specifically bind to an antigen (e.g., IL-6). It has been shown that the antigen-binding function of an antibody can be performed by portions or fragments of a full-length antibody. Examples of binding fragments encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (domain antibody) fragment (Ward et al., (1989) Nature 341:544-546; WO 90/05144 A1, each herein incorporated by reference in its entirety), which comprises a single variable domain; and (vi) an isolated complementarity determining region (CDR). The disclosure also encompasses a Fab′ fragment. Fab′ fragments can be formed by the reduction of F(ab′)₂ fragments. Fab′ is derived from F(ab′)₂; therefore, it may contain a small portion of Fc. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv). See e.g., Bird et al. (1988) Science 242:423-426; Huston et al. (1988) Proc. Natl. Acad. Sci. USA 85:5879-5883. Such single chain antibodies are also intended to be encompassed within the term “antigen-binding portion” of an antibody. In some embodiments, scFv molecules may be incorporated into a fusion protein. In some embodiments, provided herein is a single chain camelid antibody. In some embodiments, provided herein is a shark heavy chain antibody (V-NAR). See, English et al. (2020) Antibody Therapeutics, 3(1):1-9. Examples of antigen-binding portions are known in the art (Kontermann and Dubel eds., Antibody Engineering(2001) Springer-Verlag. New York. 790 pp.). In some embodiments, provided herein is a single domain antibody. In general, the term “antibody” when used herein encompasses an “antibody fragment”. An antibody fragment generally retains the antigen-binding properties of a full-length antibody.

Antibodies and antibody portions provided herein may be in multispecific (e.g., bispecific or trispecific) formats. Such multispecific molecules specifically bind to two or more different molecular targets or epitopes. In some embodiments, an antibody or an antigen-binding portion is a bispecific molecule that binds specifically to a first antigen and a second antigen, wherein the first antigen is IL-6 and the second antigen is not IL-6. In some embodiments, an antibody or an antigen-binding portion is a diabody. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. (1993) Proc. Natl. Acad. Sci. USA 90:6444-6448; Poljak et al. (1994) Structure 2:1121-1123). In some embodiments, an antibody or an antigen-binding portion is a triabody, a tetrabody, a bis-scFv or a tandem scFv. In some embodiments, an antibody or an antigen-binding portion is a dual affinity re-targeting protein.

In some embodiments, an anti-IL-6 antigen-binding portion disclosed herein is a Fab, a F(ab′)₂, a Fab′, a Fv, a scFv, a Fd, a single domain antibody, a single chain camelid antibody, a diabody, a triabody, a tetrabody or a bis-scFv.

As used herein, the terms “immunological binding” and “immunological binding properties” refer to the non-covalent interactions of the type which occur between an immunoglobulin molecule (e.g., antibody or antigen-binding portion thereof) and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity, and is equal to the dissociation constant K_{d}. (See, Davies et al. (1990) Annual Rev Biochem 59:439-473). An antibody or antigen-binding portion provided herein is said to specifically bind IL-6 when the equilibrium binding constant (K_{d}) is ≤10 μM, preferably ≤100 nM, more preferably ≤10 nM, and most preferably ≤100 pM to about 1 pM, as measured by assays such as radioligand binding assays or similar assays known to those skilled in the art.

In some embodiments, an anti-IL-6 antibody or antigen-binding portion provided herein is monovalent or bivalent and comprises a single or double chain. Functionally, the binding affinity of an antibody or antigen-binding portion may be within the range of about 10⁻⁵M to 10⁻¹² M. For example, the binding affinity of an antibody or antigen-binding portion is from about 10⁻⁶ M to 10⁻¹² M, from about 10⁻⁷ M to 10⁻¹² M, from about 10⁻⁸ M to 10⁻¹² M, from about 10⁻⁹ M, to 10⁻¹² M, from about 10⁻⁵ M, to 10⁻¹¹ M, from about 10⁻⁶ M to 10⁻¹¹ M, from about 10⁻⁷ M to 10⁻¹¹ M, from about 10⁻⁸ M to 10⁻¹¹ M, from about 10⁻⁹ M to 10⁻¹¹ M, from about 10⁻¹⁰ M to 10⁻¹¹ M, from about 10⁻⁵ M to 10⁻¹⁰ M, from about 10⁻⁶ M to 10⁻¹⁰ M, from about 10⁻⁷ M to 10⁻¹⁰ M, from about 10⁻⁸ M to 10⁻¹⁰ M, from about 10⁻⁹ M to 10⁻¹⁰ M, from about 10⁻⁵ M to 10⁻⁹ M, from about 10⁻⁶ M, to 10⁻⁹ M, from about 10⁻⁷ M to 10⁻⁹ M, from about 10⁻⁸ M to 10⁻⁹ M, from about 10⁻⁵ M to 10⁻⁸ M, from about 10⁻⁶ M to 10⁻⁸ M, from about 10⁻⁷ M to 10⁻⁸ M, from about 10⁻⁵ M to 10⁻⁷ M, from about 10⁻⁶ M to 10⁻⁷ M or from about 10⁻⁵ M to 10⁻⁶ M.

A human anti-IL-6 monoclonal antibody (PF-04236921) was described in U.S. Pat. No. 8,188,235, the content of which is incorporated herein by reference in its entirety. The human anti-IL-6 monoclonal antibody is a fully human immunoglobulin G2 monoclonal antibody that binds to human IL-6 and has a half-life of 36-51 days. In phase I trials in healthy volunteers and patients with rheumatoid arthritis (protocol B0151001,NCT00838565 and NCT01166555), intravenous and subcutaneous (SC) administration of the human anti-IL-6 monoclonal antibody (PF-04236921) was well tolerated and caused sustained suppression of C-reactive protein (CRP), a marker for inflammation that is transcriptionally controlled by IL-6. PF-04236921 has also been investigated in a phase II trial in patients with systemic lupus erythematosus (SLE; NCT01405196). While the study did not meet the primary end point, improvement was noted in the primary as well as key secondary end points with 10 mg. In 448 subjects who have received treatment with the anti-IL-6 antibody, only 2 subjects have tested positive for the presence of anti-drug antibodies. Overall, the human anti-IL-6 monoclonal antibody demonstrated desirable pharmacokinetic (PK) and pharmacodynamic (PD) properties supporting sustained target inhibition, and low incidence of immunogenicity upon single and multiple dose administration (Danese et al., Gut 2019;68:40-48; Li et al., Br J Clin Pharmacol. 2018 September; 84(9): 2059-2074.).

The amino acid and nucleic acid sequences of the human anti-IL-6 antibody (TOUR006) are provided in Table 1.

TABLE 1
Amino acid and nucleic acid sequences of human anti-IL-6 antibody

| Antibody name | Anti-IL-6 antibody (TOUR006) | |
|---|---|---|
| Domain or Region | Sequence | SEQ ID NO |
| Heavy chain | QVQLQQWGAGLLKPSETLSLTCAIYGGSFREYYW | 1 |
| (amino acid | SWIRQPPGKGLEWIGEIFHSGSTNYNPSLKSRVTIS | |
| sequence) | VDTSKNQFSLKLSSVTAADTAVYYCAREELDDFD | |
| | IWGQGTMVTVSSASTKGPSVFPLAPCSRSTSESTA | |
| | ALGCLVKDYFPEPVTVSWNSGALTSGVHTFPAVL | |
| | QSSGLYSLSSVVTVPSSNFGTQTYTCNVDHKPSNT | |
| | KVDKTVERKCCVECPPCPAPPVAGPSVFLFPPKPK | |
| | DTLMISRTPEVTCVVVDVSHEDPEVQFNWYVDG | |
| | VEVHNAKTKPREEQFNSTFRVVSVLTVVHQDWL | |
| | NGKEYKCKVSNKGLPAPIEKTISKTKGQPREPQVY | |
| | TLPPSREEMTKNQVSLTCLVKGFYPSDIAVEWESN | |
| | GQPENNYKTTPPMLDSDGSFFLYSKLTVDKSRWQ | |
| | QGNVFSCSVMHEALHNHYTQKSLSLSPGK | |
| Heavy chain CDR1 | EYYWS | 2 |
| Heavy chain CDR2 | EIFHSGSTNYNPSLKS | 3 |
| Heavy chain CDR3 | EELDDFDI | 4 |
| Heavy chain | QVQLQQWGAGLLKPSETLSLTCAIYGGSFREYYW | 5 |
| variable region | SWIRQPPGKGLEWIGEIFHSGSTNYNPSLKSRVTIS | |
| (VH) (amino acid | VDTSKNQFSLKLSSVTAADTAVYYCAREELDDFD | |
| sequence) | IWGQGTMVTVSS | |
| Heavy chain | caggtgcagctacagcagtggggcgcaggactgttgaagccttcggagacc | 6 |
| (nucleic acid | ctgtccctcacctgcgctatctatggtgggtccttcagggagtactactggagct | |
| sequence) | ggatccgccagcccccagggaaggggctggagtggattggggaaatctttca | |
| | tagtggaagcaccaactacaacccgtccctcaagagtcgagtcaccatatcag | |
| | tagacacgtccaagaaccagttctccctgaagctgagctctgtgaccgccgcg | |
| | gacacggctgtgtattactgtgcgagagaggaattagatgattttgatatctggg | |
| | gccaagggacaatggtcaccgtctcctcagcctccaccaagggcccatcggt | |
| | cttccccctggcgccctgctccaggagcacctccgagagcacagcggccctg | |
| | ggctgcctggtcaaggactacttccccgaaccggtgacggtgtcgtggaactc | |
| | aggcgctctgaccagcggcgtgcacaccttcccggctgtcctacagtcctcag | |
| | gactctactccctcagcagcgtagtgaccgtgccctccagcaacttcggcacc | |
| | cagacctacacctgcaacgtagatcacaagcccagcaacaccaaggggac | |
| | aagacagttgagcgcaaatgttgtgtcgagtgcccaccgtgcccagcaccac | |
| | ctgtggcaggaccgtcagtcttcctcttccccccaaaacccaaggacaccctc | |
| | atgatctcccggacccctgaggtcacgtgcgtggtggtggacgtgagccacg | |
| | aagaccccgaggtccagttcaactggtacgtggacggcgtggaggtgcataa | |
| | tgccaagacaaagccacgggaggagcagttcaacagcacgttccgtgtggtc | |
| | agcgtcctcaccgtcgtgcaccaggactggctgaacggcaaggagtacaagt | |
| | gcaaggtctccaacaaaggcctcccagcccccatcgagaaaacc.atctcca | |
| | aaaccaaagggcagccccgagaaccacaggtgtacaccctgcccccatccc | |
| | gggaggagatgaccaagaaccaggtcagcctgacctgcctggtcaaaggctt | |
| | ctaccccagcgacatcgccgtggagtgggagagcaatgggcagccggaga | |
| | acaactacaagaccacacctcccatgctggactccgacggctccttcttcctct | |
| | acagcaagctcaccgtggacaagagcaggtggcagcaggggaacgtcttct | |
| | catgctccgtgatgcatgaggctctgcacaaccactacacgcagaagagcctc | |
| | tccctgtctccgggtaaa | |
| Light chain (amino | DIQMTQSPSSLSASVGDRVTITCRASQGISSWLAW | 7 |
| acid sequence) | YQQKPEKAPKSLIYAASSLQSGVPSRFSGSGSGTD | |
| | FTLTISSLQPEDFATYYCQQYKSYPRTFGQGTKVE | |
| | IKRTVAAPSVFIFPPSDEQLKSGTASVVCLLNNFYP | |
| | REAKVQWKVDNALQSGNSQESVTEQDSKDSTYS | |
| | LSSTLTLSKADYEKHKVYACEVTHQGLSSPVTKS | |
| | FNRGEC | |
| Light chain CDR1 | RASQGISSWLA | 8 |
| Light chain CDR2 | AASSLQS | 9 |
| Light chain CDR3 | QQYKSYPRT | 10 |
| Light chain variable | DIQMTQSPSSLSASVGDRVTITCRASQGISSWLAW | 11 |
| region (VL) (amino | YQQKPEKAPKSLIYAASSLQSGVPSRFSGSGSGTD | |
| acid sequence) | FTLTISSLQPEDFATYYCQQYKSYPRTFGQGTKVE | |
| | IK | |
| Light chain (nucleic | gacatccagatgacccagtctccatcctcactgtctgcatctgtaggagacaga | 12 |
| acid sequence) | gtcaccatcacttgtcgggcgagtcagggtattagcagctggttagcctggtat | |
| | cagcagaaaccagagaaagcccctaagtccctgatctatgctgcatccagttt | |
| | gcaaagtggggtcccatcaaggttcagcggcagtggatctgggacagatttca | |
| | ctctcaccatcagtagcctgcagcctgaagattttgcaacttattactgccaaca | |
| | gtataaaagttaccctcggacgttcggccaagggaccaaggtggaaatcaaa | |
| | cgaactgtggctgcaccatctgtcttcatcttcccgccatctgatgagcagttga | |
| | aatctggaactgcctctgttgtgtgcctgctgaataacttctatcccagagaggc | |
| | caaagtacagtggaaggtggataacgccctccaatcgggtaactcccaggag | |
| | agtgtcacagagcaggacagcaaggacagcacctacagcctcagcagcacc | |
| | ctgacgctgagcaaagcagactacgagaaacacaaagtctacgcctgcgaa | |
| | gtcacccatcagggcctgagctcgcccgtcacaaagagcttcaacagggga | |
| | gagtgt | |

CDR1, CDR2 and CDR3 (from left to right) sequences are underlined in the heavy chain and light chain,
respectively.

Provided herein is a method of treating myasthenia gravis comprising subcutaneously administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10. In some embodiments, said antibody or antibody fragment comprises a heavy chain polypeptide comprising a polypeptide having at least about 95%, about 96%, about 97%, about 98% or about 99% identity to SEQ ID NO: 1 and a light chain polypeptide comprising a polypeptide having at least about 95%, about 96%, about 97%, about 98% or about 99% identity to SEQ ID NO: 7. In some embodiments, said antibody or antibody fragment comprises a heavy chain polypeptide comprising a polypeptide having the sequence of SEQ ID NO: 1 and a light chain polypeptide comprising a polypeptide having the sequence of SEQ ID NO: 7. In some embodiments, the anti-IL-6 antibody or an antigen-binding portion comprises human IgG2 constant regions.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89:10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4 |

Calculations of sequence homology or identity (the terms are used interchangeably herein) between sequences may be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least about 30%, preferably at least about 40%, more preferably at least about 50%, even more preferably at least about 60%, and even more preferably at least about 70%, about 75%, about 80%, about 82%, about 84%, about 85%, about 86%, about 87%, about 88%, about 89%, about 90%, about 91%, about 92%, about 93%, about 94%, about 95%, about 96%, about 97%, about 98%, about 99%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, considering the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman et al. ((1970) J. Mol. Biol. 48:444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation of the invention) is a BLOSUM 62 scoring matrix with a gap penalty of 12, a gap extend penalty of 4, and a frameshift gap penalty of 5.

In some embodiments, the percent identity between two amino acid or nucleotide sequences can be determined using the algorithm of Meyers et al. ((1989) CABIOS 4:11-17) which has been incorporated into the ALIGN program (version 2.0), using a PAM120 weight residue table, a gap length penalty of 12 and a gap penalty of 4.

In some embodiments, the anti-IL-6 antibody or antigen-binding portion provided herein is monoclonal.

In some embodiments, the anti-IL-6 antibody or antigen-binding portion provided herein is chimeric. The term “chimeric” is intended to refer to an antibody molecule, or an antigen-binding portion thereof, in which the variable domain sequences are derived from one species and at least one constant region sequence is derived from another species. For example, one or all the variable domains of the light chain(s) and/or one or all the variable domains of the heavy chain(s) of a mouse antibody (e.g., a mouse monoclonal antibody) may each be joined to a human constant region, such as, without limitation an IgG1, IgG2, or IgG4 human constant region. Examples of chimeric antibodies and suitable techniques for their generation are provided in U.S. Pat. Nos. 4,816,567; 4,975,369; and 4,816,397, each of which is incorporated herein by reference in its entirety.

In some embodiments, the anti-IL-6 antibody or antigen-binding portion provided herein is humanized. The term “humanized” is intended to refer to an antibody, or an antigen-binding portion thereof, that has been engineered to comprise one or more human framework regions in the variable domain together with non-human (e.g., mouse, rat, or hamster) CDRs of the heavy and/or light chain. In some embodiments, a humanized antibody comprises sequences that are entirely human except for the CDRs. In some embodiments, the VH domain, the VL domain, or both the VH domain and the VL domain of an anti-IL-6 antibody or antigen-binding portion provided herein comprise one or more human framework region amino acid sequences. In some embodiments, a humanized antibody comprises sequences that are entirely human except for the CDRs. Examples of humanized antibodies and suitable techniques for their generation are provided in Hwang et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and WO 90/07861, each of which is incorporated herein by reference in its entirety.

In some embodiments, humanization comprises removal of post-translational modification (PTM) sites in the variable domain sequences (e.g., in the CDR or framework sequences) of a non-human antibody. For example, one or more PTM sites in CDR sequences may be removed by substituting certain amino acid residues. In some embodiments, humanization comprises CDR grafting and back mutation.

In some embodiments, the anti-IL-6 antibody or antigen-binding portion thereof comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region is immunologically inert. In some embodiments, the immunoglobulin constant region comprises one or more mutations to reduce or prevent FcyR binding, antibody-dependent cell-mediated cytotoxicity activity, and/or complement-dependent cytotoxicity activity. In some embodiments, the immunoglobulin constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU numbering system. In some embodiments, a position of an amino acid residue in a constant region of an immunoglobulin molecule is numbered according to EU nomenclature (Ward et al., 1995 Therap. Immunol. 2:77-94).

In some embodiments, the anti-IL-6 antibody or antigen-binding portion thereof may comprise an immunoglobulin light chain constant region that is a kappa light chain constant region or a lambda light chain constant region.

In some embodiments, the anti-IL-6 antibody or antigen-binding portion thereof may comprise a human IgG4 constant region comprising the amino acid substitution S228P and a kappa light chain constant region.

Further provided herein is an immunoconjugate comprising an anti-IL-6 antibody or an antigen-binding portion linked to a therapeutic agent. In some embodiments, the therapeutic agent is a small molecule drug.

### Pharmaceutical Compositions

The anti-IL-6 antibodies and antigen-binding portions described herein (also referred to herein as “active compounds”) can be incorporated into pharmaceutical compositions suitable for administration. TOUR006 may potentially be treated through home administration (either self-administered or by caregiver or by visiting healthcare professional). Such compositions typically comprise an anti-IL-6 antibody or antigen-binding portion (or an immunoconjugate comprising said antibody or portion), and a pharmaceutically acceptable carrier, diluent or excipient. As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Provided herein is a pharmaceutical composition comprising (i) an anti-IL-6 antibody or an antigen-binding portion thereof, wherein the antibody or antigen-binding portion comprises a VH domain and a VL domain, wherein: (a) the VH domain amino acid sequence comprises HCDR1 of SEQ ID NO: 2, HCDR2 of SEQ ID NO: 3 and HCDR3 of SEQ ID NO: 4; and the VL domain amino acid sequence comprises LCDR1 of SEQ ID NO: 8, LCDR2 of SEQ ID NO: 9 and LCDR3 of SEQ ID NO: 10; and (ii) a pharmaceutically acceptable carrier, diluent or excipient.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELR (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the anti-IL-6 antibody of the present disclosure is formulated in a aqueous solution. In some embodiments, the aqueous solution comprises the anti-IL-6 antibody at a concentration ranging from about 50 mg/mL to about 150 mg/mL. In some embodiments, the aqueous solution comprises the anti-IL-6 antibody at a concentration of about 50 mg/mL, about 55 mg/mL, about 60 mg/mL, 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL about 120 mg/mL, about 125 mg/mL, about 130 mg/mL, about 135 mg/mL, about 140 mg/mL, about 145 mg/mL, or about 150 mg/mL.

In some embodiments, a buffer is selected from phosphate buffers, histidine, sodium citrate, HEPES, Tris, Bicine, glycine, N-glycylglycine, sodium acetate, sodium carbonate, glycylglycine, lysine, arginine, sodium phosphate, and any combination thereof. Exemplary concentrations of buffers for formulations of the present disclosure are from about 5 mM to about 100 mM, about 50 mM, about 10 mM to about 40 mM, or about 20 mM. In some embodiments, histidine is included at about 5 mM, about 10 mM, about 20 mM, about 30 mM, about 40 mM, or about 50 mM.

In some embodiments, a sweetening agent is selected from sucrose and saccharin. In some embodiments, sucrose is included at about 50 mg/mL, about 60 mg/mL, about 80 mg/mL, about 90 mg/mL, or about 100 mg/mL.

In some embodiments, a tonicity adjusting agent is selected from sodium chloride, potassium chloride, dextrose, mannitol, glycerin, sorbitol, and any combination thereof. In some embodiments, mannitol is included at about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL.

In some embodiments, a chelating agents is selected from ethylenediaminetetraacetic acid (EDTA), disodium edetate, calcium EDTA, and any combination thereof. In some embodiments, EDTA is included at about 0.01 mg/mL, about 0.02 mg/mL, 0.03 mg/mL, about 0.04 mg/mL, 0.05 mg/mL, about 0.06 mg/mL, 0.07 mg/mL, about 0.08 mg/mL, about 0.09 mg/mL, or 0.10 mg/mL.

In some embodiments, a surfactant is selected from polysorbate 80, sodium lauryl sulfate (SDS), Tween 80, and any combination thereof. In some embodiments, polysorbate 80 is included at about 0.1 mg/mL, about 0.2 mg/mL, 0.3 mg/mL, about 0.4 mg/mL, 0.5 mg/mL, about 0.6 mg/mL, 0.7 mg/mL, about 0.8 mg/mL, about 0.9 mg/mL, or about 1.0 mg/mL.

In some embodiments, the formulation has a pH value ranging from about 5.0 to about 8.0. In some embodiments, the formulation has a pH value of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

In some embodiments, TOUR006 is formulated at a concentration of 85 mg/mL with 20 mM histidine, 63.2 mg/mL sucrose, 16.8 mg/mL mannitol, 0.05 mg/mL EDTA, and 0.2 mg/mL polysorbate 80, pH 5.8. After reconstitution with water for injection, each single use vial contains 106 mg of TOUR006 in 1.25 mL of aqueous solution.

The pharmaceutical compositions provided herein can be included in a container, pack, or dispenser together with instructions for administration.

### Uses of Antibodies

Provided herein are methods and uses of the anti-IL-6 antibodies, anti-IL-6 antigen-binding portions, immunoconjugates and pharmaceutical compositions described herein for providing a therapeutic benefit to a subject with a condition associated with IL-6 expression. In some embodiments, the condition is myasthenia gravis.

In some embodiments, the methods described herein further comprise a step of treating a subject with an additional form of therapy. In some embodiments, the additional form of therapy comprises administering one or more therapeutic agent in addition to the said anti-IL-6 antibody or antibody fragment as described herein. The therapeutic agents include, but are not limited to, a second antibody (e.g., an anti-IL-1 antibody, anti-IGF-1 receptor antibody, anti-VEGF antibody, and/or anti-IL17a antibody), a soluble receptor (e.g., soluble IL-1 receptor, soluble TNF-alpha receptor), an anti-inflammatory agent (e.g., paclitaxel, docetaxel, cisplatin, doxorubicin, prednisone, mitomycin, progesterone, tamoxifen, or fluorouracil), or other common myasthenia gravis medication (e.g., acetylcholinesterase inhibitors; glucocorticoids or nonsteroidal immunosuppressive and immunomodulatory agents; or therapeutic plasma exchange or intravenous immune globulin.)

Provided herein is an anti-IL-6 antibody or an anti-IL-6 antigen-binding portion, an immunoconjugate or a pharmaceutical composition described herein, for use as a medicament.

As used herein, the term “effective amount” or “therapeutically effective amount” refers to the amount of a pharmaceutical agent, e.g., an anti-IL-6 antibody or an antigen-binding portion thereof, which is sufficient to reduce or ameliorate the severity and/or duration of a disorder, e.g., myasthenia gravis, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). In some embodiments, the therapeutically effective dose of said anti-IL-6 antibody or antibody fragment is effective to change one or more biomarkers of IL-6 mediated signaling including, but not limited to, total sIL-6R, total IL-6, C-reactive protein (CRP), an/or autoantibodies, for unexpectedly prolonged periods of time.

As used herein, the terms “treat,” “treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition or symptoms associated therewith be completely eliminated.

As used herein, “pre-treatment” means prior to the first administration of an anti-anti-IL-6 antibody according to the methods described herein. Pre-treatment does not exclude, and often includes, the prior administration of treatments other than an anti-IL-6 antibody.

As used herein, “post-treatment” means after the administration of an anti-IL-6 antibody according to the methods described herein. Post-treatment includes after any administration of an anti-IL-6 antibody at any dosage described herein. Post-treatment also includes after the treatment phase of an anti-IL-6 antibody.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are well known in the art (Ledermann et al., 1991, Int. J. Cancer 47:659-664; Bagshawe et al., 1991, Antibody, Immunoconjugates and Radiopharmaceuticals 4:915-922). Specific dosages may be indicated herein or in the Physician's Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g., whole antibody, fragment) and the nature of any detectable label or other molecule attached to the antibody.

A typical antibody dose will be in the range 100 μg to 1 g for systemic applications, and 1 μg to 1 mg for intradermal injection. In one embodiment, an initial higher loading dose, followed by one or more lower doses, may be administered. In another embodiment, an initial lower loading dose, followed by one or more higher doses, may be administered. In some embodiments, the antibody is a whole antibody, e.g., the IgG1, IgG2 or IgG4 isotype. This is a dose for a single treatment of an adult subject, which may be proportionally adjusted for children and infants, and also adjusted for other antibody formats in proportion to molecular weight. Treatments may be repeated at daily, twice-weekly, weekly or monthly intervals, at the discretion of the physician. The treatment schedule for a subject may be dependent on the pharmacokinetic and pharmacodynamic properties of the antibody composition, the route of administration and the nature of the condition being treated. In some embodiments, the dosing of the present disclosure comprises an amount of at least about 10 mg, or at least about 20 mg, or at least about 30 mg, or at least about 40 mg, at least about 50 mg, at least about 60 mg, at least about 70 mg, at least about 80 mg, at least about 90 mg, or at least about 100 mg of the anti-IL-6 antibody or antibody fragment.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above. In some embodiments, the dosing schedules for the anti-IL-6 antibody or antibody fragment is once every 4 or 8 weeks up to about 52 total weeks.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

### Myasthenia Gravis

As used herein, the term “myasthenia gravis (MG)” refers to is an autoimmune disease characterized by autoantibodies directed against epitopes of the post-synpatic muscle membrane, including the nicotinic acetylcholine receptor (AChR) and the muscle-specific tyrosine kinase receptor (MuSK), and complement-mediated destruction of the post junctional membrane. Around 10-20% of MG patients do not have AChR antibodies (seronegative), of whom some have antibodies to a membrane-linked MuSK. Clinical manifestations include fluctuating weakness of ocular, bulbar, respiratory and limb muscles.

As used herein, the term “MG Foundation of America (MGFA) Clinical Classification” refers to a common clinical classification tool for MG. This is a 5-stage classification (I to V), with a higher class indicating more severe disease. (Jaretzki et al, Neurol. 2000; 55:16-23). Typically moderate to severe is classified as class II-class IVa. Table 2 provides the MGFA clinical classification which divides MG presentations into different classes by clinical features with increasing severity of diseases.

TABLE 2
Myasthenia Gravis Foundation of America (MGFA) clinical
classification of MG

| Class | |
|---|---|
| (I-V) | Clinical features |
| Class I | Any ocular muscle weakness. May have weak eye closure. |
| | All other muscle strength is normal. |
| Class II | Mild weakness affecting non-ocular muscles May also have |
| | any ocular weakness |
| IIa | Predominantly affecting limb or axial muscles or both |
| | May also have oropharyngeal muscle weakness (less than |
| | limb/axial) |
| IIb | vs |
| | Predominantly affecting oropharyngeal or respiratory |
| | muscles or both May also have limb or axial or both muscle |
| | involvement (less or equal to oropharyngeal/respiratory) |
| Class III | Moderate weakness affecting non-ocular muscles May also |
| | have any ocular weakness |
| IIIa | Predominantly affecting limb or axial muscles or both |
| | May also have oropharyngeal muscle weakness (less than |
| | limb/axial) |
| IIIb | vs |
| | Predominantly affecting oropharyngeal or respiratory |
| | muscles or both May also have limb or axial or both muscle |
| | involvement (less or equal to oropharyngeal/respiratory) |
| Class IV | Severe weakness affecting non-ocular muscles May also have |
| | any ocular weakness |
| IVa | Predominantly affecting limb or axial muscles or both May |
| | also have oropharyngeal muscle weakness (less than |
| | limb/axial) |
| IVb | vs |
| | Predominantly affecting oropharyngeal or respiratory |
| | muscles or both May also have limb or axial or both muscle |
| | involvement (less or equal to oropharyngeal/respiratory) |
| Class V | Defined by intubation with or without mechanical ventilation |
| | (except when this is employed during routine post-op |
| | management) |

The major pathophysiology leading to MG is the abnormal production of IgG autoantibodies directed toward nicotinic acetylcholine receptor (AChR) or muscle specific kinase (MuSK) protein and both can be measured using standard methods known in the art, such as radioimmunoprecipitation, ELISA and cell-based assays.

Several methods of characterizing MG and treatment efficacy are established.

As used herein, the term “Myasthenia Gravis Activities of Daily Living (MG-ADL) score” refers to an 8-item patient-reported scale developed to assess MG symptoms and their effects on daily activities (Wolfe et al, Neurology, 1999 Apr. 22; 52(7):1487-9). Items are linearly scored and not weighted, with each item ranging from 0 to 3 for a total score range of 0 to 24. Table 3 describes the eight items of MG-ADL profile.

TABLE 3
MG-ADL profile

| Symptom | | 0 | 1 | 2 | 3 |
|---|---|---|---|---|---|
| 1. | Talking | Normal | Intermittent | Constant | Difficult to |
| | | | slurring of | slurring or | understand |
| | | | nasal speech | nasal, but can | speech |
| | | | | be understood | |
| 2. | Chewing | Normal | Fatigue with | Fatigue with | Gastric |
| | | | solid food | soft food | tube |
| 3. | Swallowing | Normal | Rare episode | Frequent | Gastric |
| | | | of choking | choking | tube |
| | | | | necessitating | |
| | | | | changes in diet | |
| 4. | Breathing | Normal | Shortness of | Shortness of | Ventilator |
| | | | breath with | breath at rest | dependence |
| | | | exertion | | |
| 5. | Impairment of | None | Extra effort, | Rest periods | Cannot do |
| | ability to brush | | but no rest | needed | one of these |
| | teeth or comb | | periods | | functions |
| | hair | | needed | | |
| 6. | Impairment of | None | Mild, | Moderate, | Severe, |
| | ability to arise | | sometimes | always | request |
| | from a chair | | uses arms | uses arms | assistance |
| 7. | Double vision | None | Occurs, but | Daily, but not | Constant |
| | | | not daily | constant | |
| 8. | Eyelid droop | None | Occurs, but | Daily, but not | Constant |
| | | | not daily | constant | |

The total MG-ADL score ranges from 0 to 24, with a higher score indicating more disability. A 2-point reduction in MG-ADL score from baseline indicates clinical improvement (Muppidi, Ann N Y Acad Sci, 2012. 1274: p. 114-9; Muppidi, Muscle Nerve. 2011 November; 44(5):727-31).

As used herein, the term “Quantitative Myasthenia Gravis score (QMG)” refers to a 13-item direct physician assessment scoring system that quantifies disease severity based on impairments of body functions and structures. Each item is quantitatively assessed and scored from 0 to 3 (where 3 represents the most severe), providing a total QMG score ranging from 0 to 39. (Barnett, J Clin Neuromuscul Dis. 2012; 13(4):201-5). A 3-point change in the total score is considered clinically relevant. Table 4 provides the quantitative myasthenia gravis score items and scoring.

TABLE 4
Quantitative myasthenia gravis score items and scoring

| | | | | |
|---|---|---|---|---|
| Grade (score) | None (0) | Mild (1) | Moderate (2) | Severe (3) |

Test item

| | | | | |
|---|---|---|---|---|
| 1. Ptosis | 61 | 11-60 | 1-10 | Spontaneous |
| (upward gaze), s | | | | |
| 2. Diplopia | 61 | 11-60 | 1-10 | Spontaneous |
| (lateral gaze), | | | | |
| R or L, s | | | | |
| 3. Eyelid closure | Normal | Complete, | Complete, | Incomplete |
| | | some | no | |
| | | resistance | resistance | |
| 4. Dysarthria with | None | 30-49 | 10-29 | 9 or less |
| counting 1-50 | | | | |
| 5. Swallowing 4 oz. | Normal | Mild | Severe | Unable |
| water | | cough, | cough/ | |
| | | throat | choking | |
| | | clearing | | |
| 6. Vital capacity, % | ≥80 | 65-79 | 50-64 | <50 |
| predicted | | | | |
| 7. Right arm held | 240 | 90-239 | 10-89 | 0-9 |
| outstretched at 90°, s | | | | |
| 8. Left arm held | 240 | 90-239 | 10-89 | 0-9 |
| outstretched at 90°, s | | | | |
| 9. Right hand grip, | ≥45/≥30 | 15-44/ | 5-4/5-9 | 0-4/0-4 |
| kgw, male/female | | 10-29 | | |
| 10. Left hand grip, | ≥35/≥25 | 15-34/ | 5-4/5-9 | 0-4/0-4 |
| kgw, male/female | | 10-24 | | |
| 11. Head lift 45° | 120 | 30-119 | 1-29 | 0 |
| supine, s | | | | |
| 12. Right leg held | 100 | 31-99 | 1-30 | 0 |
| outstretched at | | | | |
| 45° supine, s | | | | |
| 13. Left leg held | 100 | 31-99 | 1-30 | 0 |
| outstretched at | | | | |
| 45° supine, s | | | | |

As used herein, the term “MG Composite (MGC) score” refers to an outcome measure of signs and symptoms for patients with MG, with a higher score indicating more severe disease and a 3-point change being of clinical relevance (Burns et al, Neurology. 2010 May 4; 74(18):1434-40). The scale tests 10 items, with individual items being weighted differently. The overall score ranges from 0 to 50. Table 5 provides the MGC scale.

TABLE 5
MG composite scale

| | | | | |
|---|---|---|---|---|
| 1. Ptosis, upward | >45 | 11-45 | 1-10 | Immediate = |
| ease (physician | seconds = | seconds = 1 | seconds = 2 | 3 |
| examination) | 0 | | | |
| 2. Double vision | >45 | 11-45 | 1-10 | Immediate = |
| on lateral Gaze, | seconds = | seconds = 1 | seconds = 3 | 4 |
| left or right | 0 | | | |
| (physician | | | | |
| examination) | | | | |
| 3. Eye closure | Normal = | Mild | Moderate | Severe |
| (physician | 0 | weakness | weakness | weakness |
| examination) | | (can be | (can be | (unable to |
| | | forced | forced | keep |
| | | open with | open | eye |
| | | effort) = 0 | easily) = 1 | closed) = 2 |
| 4. Talking | Normal = | Intermittent | Constant | Difficult to |
| (patient history) | 0 | slurring or | slurring or | understand |
| | | nasal | nasal but | speech = 6 |
| | | speech = 2 | can be | |
| | | | understood = | |
| | | | 4 | |
| 5. Chewing | Normal = | Fatigue with | Fatigue with | Gastric |
| (patient history) | 0 | solid food = 2 | soft food = 4 | tube = 6 |
| 6. Swallowing | Normal = | Rare episode | Frequent | Gastric |
| (patient history) | 0 | of choking | trouble | tube = 6 |
| | | or trouble | swallowing, | |
| | | swallowing = | for example | |
| | | 2 | necessitating | |
| | | | change in | |
| | | | diet = 5 | |
| 7. Breathing | Normal = | Shortness of | Shortness of | Ventilator |
| (thought to be | 0 | breath with | breath at | dependence = |
| caused by MG) | | exertion = 2 | rest = 4 | 9 |
| 8. Neck flexion | Normal = | Mild | Moderate | Severe |
| or extension | 0 | weakness = 1 | weakness | weakness= 4 |
| (weakest) | | | (i.e., ~50% | |
| (physician | | | weak, | |
| examination) | | | ±15%) = 3 | |
| 9. Shoulder | Normal = | Mild | Moderate | Severe |
| abduction | 0 | weakness = 2 | weakness | weakness = 5 |
| (physician | | | (i.e., ~50% | |
| examination) | | | weak, | |
| | | | ±15%) = 4 | |
| 10. Hip flexion | Normal = | Mild | Moderate | Severe |
| (physician | 0 | weakness = 2 | weakness | weakness = 5 |
| examination) | | | (i.e., ~50% | |
| | | | weak, | |
| | | | ±15%) = 4 | |

As used herein, the term “Myasthenia Gravis Quality of Life 15 Scale (MG-QOL 15r)” refers to a survey designed to assess aspects of life related to myasthenia gravis. (Burns et al, Muscle Nerve. 2008 August;38(2):957-63). There is appropriate correlation between the 15 items of MG-QOL 15r and other MG-specific scales (e.g., MGC and MG-ADL). The MG-QOL 15r has construct validity in the clinical practice setting and represents an efficient and valuable to for assessing quality of life for patient with MG. However the magnitude of change required to indicate improvement or worsening is variable and depends on MG severity. The MG-QOL 15r can be completed by the patient or administered by the physician or trained clinic personnel or study coordinator. Table 6 provides the 15 items of MG-QOL 15r.

TABLE 6
Myasthenia Gravis Quality of Life 15 Scale

| | Not at all | Somewhat | Very much |
|---|---|---|---|
| Questions | 0 | 1 | 2 |
| 1. 1 am frustrated by my MG | | | |
| 2. I have trouble with my eyes | | | |
| because of my MG (e.g. double | | | |
| vision) | | | |
| 3. I have trouble eating because of | | | |
| MG | | | |
| 4. I have limited my social activity | | | |
| because of my MG | | | |
| 5. My MG limits my ability to | | | |
| enjoy hobbies and fun activities | | | |
| 6. I have trouble meeting the needs | | | |
| of my family because of my MG | | | |
| 7. I have to make plans around my | | | |
| MG | | | |
| 8. I am bothered by limitations in | | | |
| performing my work (include work | | | |
| at home) because of my MG | | | |
| 9. I have difficulty speaking due to | | | |
| MG | | | |
| 10.10. I have lost some personal | | | |
| independence because of my MG | | | |
| (e.g. driving, shopping, running | | | |
| errands) | | | |
| 11. I am depressed about my MG | | | |
| 12. I have trouble walking due to | | | |
| MG | | | |
| 13. I have trouble getting around | | | |
| public places because of my MG | | | |
| 14. I feel overwhelmed by my MG | | | |
| 15. I have trouble performing my | | | |
| personal grooming needs due to MG | | | |

These classifications and measures as described herein provide a consistent methodology for MG diagnosis. For example, a human who has a diagnosis of moderate to severe generalized myasthenia gravis, is anti-AChR and/or anti-MuSK autoantibody-positive, and/or has a MGFA Class II to IVa, and/or a QMG score of at least 12, and/or has a MG-ADL score of at least 4.

These outcome measures are being used as primary endpoints in clinical trials for new MG therapies. Efficacy of treatment for MG can be determined by a reduction in serum concentrations of anti-AChR antibody, and/or a reduction in the MG-ADL score, and/or a reduction in the QMG, and/or a reduction in the MGC score, and/or a reduction in MG-QOL 15r score.

As used herein, the term “C-reactive protein (CRP)” refers to a marker of inflammation. CRP levels increase in response to inflammation, and can be measured with a hsCRP (high-sensitivity C-reactive protein) test. The pre-treatment hsCRP of the patients is typically greater than 2 mg/L.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

The disclosure will be further clarified by the following examples, which are intended to be purely exemplary of the disclosure and in no way limiting.

### EXAMPLES

### Example 1: Phase 2 Clinical Evaluation of Human Anti-IL6 Antibody in Patients With Myasthenia Gravis

This is a Phase 2 randomized, double-masked, placebo-controlled, parallel arm study to compare the efficacy and safety of 2 different doses of subcutaneously administered TOUR006 versus placebo (PBO) in the first-line treatment of patients with myasthenia gravis (MG).

The criteria for a patient to be eligible to participate in this study may include, but is not limited to,
is positive for anti-Acetylcholine Receptor (AChR), or anti-muscle specific kinase (MuSK) antibody.
has seronegative MG.
has elevated high-sensitivity C-reactive protein (hsCRP) of at least 2 mg/L, 5 mg/L, or 10 mg/L.
has elevated serum IL-6.
has elevated red blood cell distribution width (RDW) of at least 13% or in the highest quartile.
has Myasthenia Gravis Foundation of America (MGFA) class II.
has MGFA class III.
has MGFA class IV.
has a Quantitative Myasthenia Gravis (QMG) score of at least 12.
has a Myasthenia Gravis Activities of Daily Living (MG-ADL) score of at least 4
is naïve to a pharmacologic disease-modifying MG treatment (e.g., acetylcholinesterase inhibitor therapy).
has no or inadequate response to efgartigimod therapy.
has a response to efgartigimod therapy followed by a relapse.
has no or inadequate response to glucocorticoid therapy.
has a response to glucocorticoid therapy followed by a relapse.
has no or inadequate response to intravenous immunoglobulin (IVIG) therapy.
has a response to IVIG therapy followed by a relapse.
has no or inadequate response to plasmapheresis therapy.
has a response to plasmapheresis therapy followed by a relapse.
has no or inadequate response to non-steroidal immunosuppressant therapy (e.g., mycophenolate mofetil, cyclophosphamide, cyclosporine, azathioprine, methotrexate, and tacrolimus).
has a response to non-steroidal immunosuppressant therapy followed by a relapse.
has no or inadequate response to thymectomy.
has a response to thymectomy followed by a relapse.

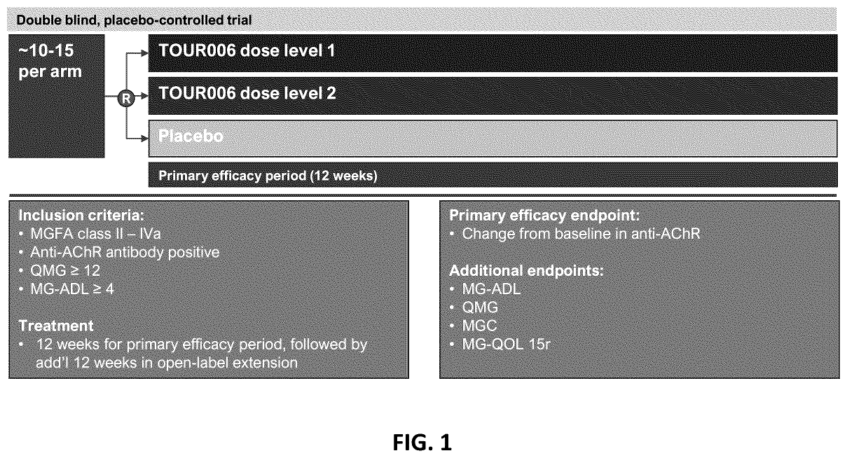

The study schematic is shown in FIG. 1. The study will consist of a 12-week primary treatment period that is placebo-controlled and a 12-week open-label extension period in which all patients receive TOUR006. The primary treatment period might be increased to 24 weeks. For Period A, approximately 45 eligible patients (10-15 per treatment arm) will be randomized in a 1:1:1 ratio to receive SC treatment with TOUR006(dose level 1), TOUR006 (dose level 2) or placebo. The patient sample sizes might be adjusted based upon ongoing power calculations. Dose level 1 will be 50 mg loading dose ×1, followed 4 weeks later by 20 mg every 4 weeks for a total of 12 weeks primary treatment. Dose level 2 will be 20 mg loading dose×1, followed 4 weeks later by 10 mg every 4 weeks for a total of 12 weeks primary treatment.

After completion of primary efficacy period, patients will be offered the option of continuing their study participation by entering extension period. All patients in extension period will be treated with TOUR006 every 4 weeks (up to a maximum cumulative exposure of 24 weeks across primary efficacy and extension period). Patients who have received TOUR006 in primary efficacy period will continue receiving the same dose level of TOUR006 with which they have previously been treated. Patients who have received placebo in extension period will be randomized to receive one of the two different dose levels of TOUR006. After the last treatment visit (Week 32), patients will undergo additional visits for further follow-up until Week 68.

Alternative dosing regimens might be considered. For example, dose level 1 will be 50 mg every 8 weeks for a total of 24 weeks treatment and dose level 2 will be 20 mg every 8 weeks for a total of 24 weeks treatment. The primary efficacy period in the Phase 2 trial will evaluate the efficacy outcomes after 12 weeks of treatment, and then patients will continue with an additional 12 weeks of treatment in the Extension Period afterwards. Additional dosing regimens include 20, 25, 30, or 50 mg every 12 week for a total of 24 weeks treatment. Moreover, additional loading dosing regimens might be considered. For example, a loading dosing regimen will be 50 mg loading dose ×1, followed 4 weeks later by 20 mg, 25 mg or 30 mg every 12 weeks; 30 mg loading dose ×1, followed 4 weeks later by 20 mg, 25 mg or 30 mg every 12 weeks; or 25 mg loading dose ×1, followed 4 weeks later by 10 mg, 20 mg or 25 mg every 12 weeks.

Clinically relevant parameters include, but not limited to, serum concentrations of anti-AChR antibody, MG-ADL score, QMG score, MGC score, and MG-QOL 15r score. Details of these clinically relevant parameters are described in US2022014494 and US20220144941, the contents of each of which are hereby expressly incorporated by reference in their entirety for any purpose.

The primary efficacy end point is the reduction in serum concentrations of anti-AChR antibody.

The additional efficacy end points include a reduction in the MG-ADL score, and/or a reduction in the QMG, and/or a reduction in the MGC score, and/or MG-QOL 15r score. For example, clinical improvements may include a reduction in the MG-ADL score by 2 points from baseline, and/or a reduction in the QMG by 3 points from baseline, and/or a reduction in the MGC score by 3 points from baseline, and/or a reduction in MG-QOL 15r score by 4 points from baseline.

The treatment result may be achieved within 16 weeks, 12 weeks, 8 weeks, or 4 weeks. The treatment result may also be achieved during a long-term treatment (e.g. more than 24 weeks, more than 48 weeks, more than 72 weeks, or more than 96 weeks after treatment initiation).

The efficacy effect of the treatment may be sustained for at least 4 weeks, 8 weeks, 12 weeks, 16 weeks, 24 weeks, 36 weeks, 48 weeks, or 72 weeks after the last dose has been administered. The probability of relapse (i.e., loss of proptosis response or CAS response or diplopia response) is less than 40%, 30%, 20%, 15%, 10%, or 5%.

The study consists of three phases: (i) screening and randomization, (ii) treatment, and (iii) follow-up. During the screening, each potential subject will provide informed consent before starting any study-specific procedures. The randomization of subjects to study groups will be performed centrally by an Interactive Web-Response System (IWRS) using a randomization scheme reviewed and approved by an independent statistician. During the treatment period, randomized subjects will be provided the treatment and assessment according to the protocol. Follow-up will occur 8 weeks, 12 weeks, 16 weeks, 24 weeks, or 36 weeks following termination of the treatment.

### Example 2: Phase 3 Clinical Evaluation of Human Anti-IL6 Antibody in Patients With Myasthenia Gravis

This is a Phase 3 randomized, double-masked, placebo-controlled study of the clinical efficacy of subcutaneously administered TOUR006 versus placebo (PBO) in the first-line treatment of patients with myasthenia gravis (MG).

Eligible patents may have a confirmed positive record of autoantibodies against AChR or MuSK; have MGFA Class II to IVa; have a MG-ADL score of at least 4; or have a QMG score of at least 12.

The study includes one active TOUR006 treatment arm and one placebo arm. The total sample size of the study could be approximately 150 study participants (about 75 for each arm). The efficacy treatment period is 24 weeks.

The primary efficacy end point is the change from baseline in MG-ADL score and the additonal efficacy end points include the reduction in serum concentrations of anti-AChR antibody, the change from baseline in QMG, MGC, and MG-QOL 15r scores.

### Example 3. PK/PD Modeling of TOUR006

The purpose of this study is to inform dosing parameters for the treatment of MG with TOUR006 using pharmacokinetic/pharmacodynamic (PK/PD) based simulations.

C-reactive protein (CRP) is directly downstream of IL-6 signal transduction, and irrelevant if ligand or receptor is blocked. There is tightly linked temporal association between CRP and IL-6 pathway. Thus, serum CRP is a promising pharmacology marker of IL-6 pathway activity. Using CRP as marker to identify what level of IL-6 pathway suppression associated with the dosing regimen would inform PD goal of TOUR006 for the treatment of MG.

Population modeling analyses were conducted using nonlinear mixed effects modeling. The CRP data for the modeling came from a multiple dose study of TOUR006 in rheumatoid arthritis (RA) patients receiving background methotrexate (NCT00838565) and the clinical investigation of tocilizumab in RA patients (Paccaly et al., J Clin Pharmacol. 2021 January; 61(1): 90-104; Xu et al., J. of Clinical Pharma., 2021, 61(5): 714-724.). The assumption of the modeling is that the background inflammatory state of MG is similar to RA. The published observations of CRP levels in MG are consistent with the model's assumptions. For example, mean CRP concentrations in patients with MG are observed to less than 10 mg/L. (Jiang et al., Int J Clin Exp Med 2019; 12(6):6789-6797; Janik et al., Oncotarget. 2017 Jul. 18; 8(29): 47090-47102.).

The PK/PD model explored two subpopulations with relatively less severe and more severe inflammation:
Population A: Baseline CRP>2 mg/L to 10 mg/L
Population B: Baseline CRP>10 mg/L.

The PK/PD based simulations were performed for the dosing scenarios given in Table 7.

TABLE 7
Dosing regimen

50 mg LD, followed by 20 mg Q4W starting at 4 weeks
20 mg LD, followed by 10 mg Q4W starting at 4 weeks
50 mg Q8W
20 mg Q8W
50 mg Q12W
30 mg Q12W
20 mg Q12W
50 mg LD, followed by 50 mg Q12W starting at 4 weeks
20 mg LD, followed by 30 mg Q12W starting at 4 weeks
Note: “LD” refers to loading dose; “Q” herein refers to “every”; and “W” refers to “week”

The simulation results were calculated for 4, 8, 12, 16, 20, and 24 weeks from the first dose.

The CRP suppression goal is at least 90% decrease from baseline (based upon CRP effects observed from Tocilizumab 8 mg/kg IV q4 weeks in RA). The simulations modeled what percentage of patients attained the CRP suppression goal for a given TOUR006 regimen. Any patient with CRP suppression of less than 2 mg/L after 7 days of treatment was considered to have at least 90% suppression. This was done to avoid ceiling effects (<2 mg/L is entering into the normal range).

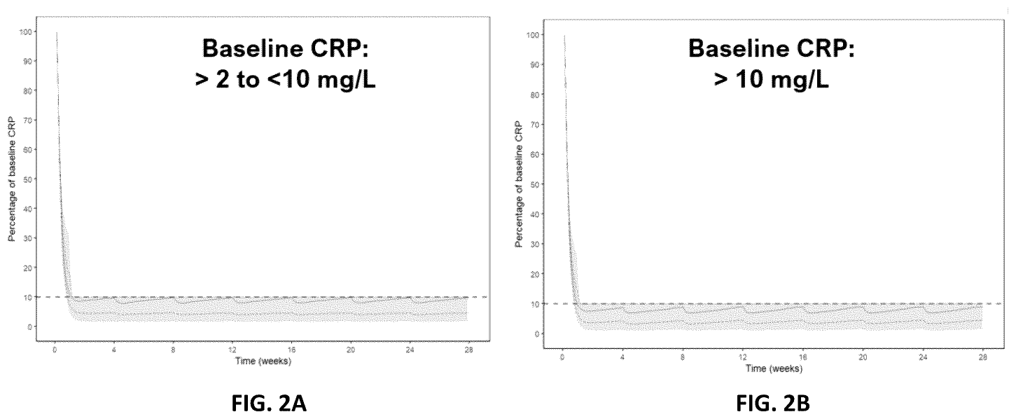

As shown in FIG. 2A and FIG. 2B, the PK/PD modeling predicts that nearly all patients, including population A (FIG. 2A) and population B (FIG. 2B), rapidly attain the CRP suppression goal (>90% decrease) under the dose regimen of 50 mg LD, followed by 20 mg Q4W starting at 4 weeks.

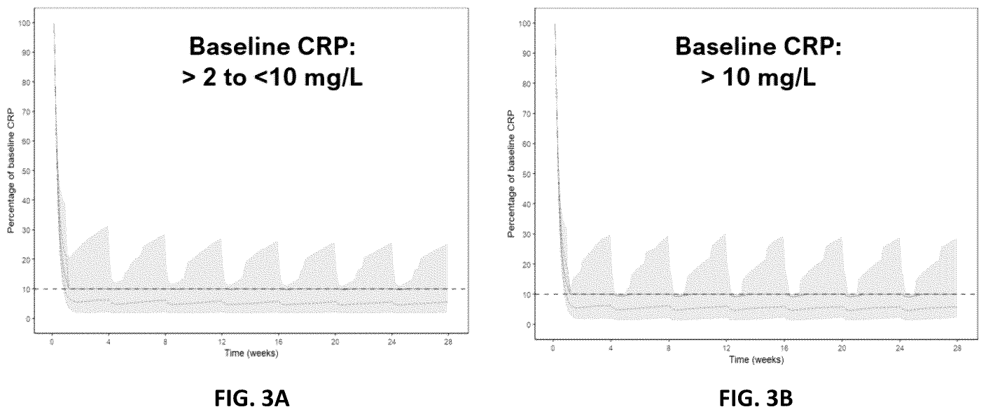

As shown in FIG. 3A and FIG. 3B, the PK/PD modeling predicts that most patients (˜90%), including population A (FIG. 3A) and population B (FIG. 3B), rapidly attain the CRP suppression goal (>90% decrease) under the dose regimen of 20 mg LD, followed by 10 mg Q4W starting at 4 weeks, although with incomplete population coverage relative to the dose regimen of 50 mg LD, followed by 20 mg Q4W starting at 4 weeks.

Further, the PK/PD modeling predicts rapid and robust CRP suppression for both dose regimens −50 mg LD, followed by 20 mg Q4W starting at 4 weeks, and 20 mg LD, followed by 10 mg Q4W starting at 4 weeks. Table 8 and Table 9 provide the percentage of patients with at least 90% CRP suppression over the treatment period.

TABLE 8
Percent of patients with ≥90% CRP suppression under the dose
regimen of 50 mg LD, followed by 20 mg Q4W starting at 4 weeks

| | | Week | Week | Week | Week | Week | Week |
|---|---|---|---|---|---|---|---|
| Baseline CRP | | 4 | 8 | 12 | 16 | 20 | 24 |
| >2 to ≤10 | mg/L | 94% | 94% | 94% | 94% | 94% | 94% |
| >10 | mg/L | 97% | 96% | 96% | 95% | 95% | 95% |

TABLE 9
Percent of patients with ≥90% CRP suppression under the dose
regimen of 20 mg LD, followed by 10 mg Q4W starting at 4 weeks

| | | Week | Week | Week | Week | Week | Week |
|---|---|---|---|---|---|---|---|
| Baseline CRP | | 4 | 8 | 12 | 16 | 20 | 24 |
| >2 to ≤10 | mg/L | 89% | 90% | 90% | 91% | 91% | 91% |
| >10 | mg/L | 89% | 89% | 89% | 89% | 89% | 90% |

Additionally, the PK/PD modeling predicts that less frequent dosing regimens achieve the CRP suppression goal of at least 90% decrease from baseline within the 24 weeks treatment period. Tables 10 to 12 provide the percentage of patients with at least 90% CRP suppression over the treatment period under the less frequent dosing regimens.

TABLE 10
Percent of patients with ≥90% CRP suppression
under the dose regimen of 50 mg Q8W

| | | Week | Week | Week | Week | Week | Week |
|---|---|---|---|---|---|---|---|
| Baseline CRP | | 4 | 8 | 12 | 16 | 20 | 24 |
| >2 to ≤10 | mg/L | 98% | 95% | 98% | 97% | 98% | 97% |
| >10 | mg/L | 95% | 93% | 97% | 95% | 98% | 95% |

TABLE 11
Percent of patients with ≥90% CRP suppression
under the dose regimen of 20 mg Q8W

| | | Week | Week | Week | Week | Week | Week |
|---|---|---|---|---|---|---|---|
| Baseline CRP | | 4 | 8 | 12 | 16 | 20 | 24 |
| >2 to ≤10 | mg/L | 94% | 88% | 94% | 93% | 95% | 93% |
| >10 | mg/L | 91% | 80% | 93% | 85% | 94% | 87% |

TABLE 12
Percent of patients with ≥90% CRP suppression
under the dose regimen of 50 mg Q12W

| | | Week | Week | Week | Week | Week | Week |
|---|---|---|---|---|---|---|---|
| Baseline CRP | | 4 | 8 | 12 | 16 | 20 | 24 |
| >2 to ≤10 | mg/L | 99% | 99% | 94% | 100% | 98% | 94% |
| >10 | mg/L | 99% | 96% | 94% | 99% | 96% | 94% |

The PK/PD modeling predicts effective dosage arms for MG Phase 2 trial. Specifically, a dosing regimen of 50 mg LD, followed by 20 mg Q4W starting at 4 weeks is predicted to result in 94-98% of patients to achieve target CRP suppression in both the moderate inflammation (i.e., baseline CRP 2 to 10 mg/L) and severe inflammation (i.e., baseline CRP of >10 mg/L) populations. A dosing regimen of 20 mg LD, followed by 10 mg Q4W starting at 4 weeks is predicted to result in ˜90% of patients to achieve target CRP suppression in both the moderate inflammation and severe inflammation populations. Both regimens are predicted to achieve rapid (i.e., in 2 weeks or less) suppression of CRP by ≥90% from baseline.

The PK/PD modeling also predicts that less frequent dosing regimens offer opportunity for robust CRP suppression while further reducing drug administration burden for patients. Specifically, the less frequent dosing regimens including 50 mg Q8W and 50 mg Q12W regimens are both predicted to offer CRP suppression effects similar to the 50 mg LD, followed by 20 mg Q4W starting at 4 weeks regimen. The 20 mg Q8W regimen is predicted to offer CRP suppression effects similar to the regimen of 20 mg LD, followed by 10 mg Q4W starting at 4 weeks.

The PK/PD modeling results predict that less frequent or lower dose regimens offer similar CRP suppression effects, particularly if a loading dose is used.

The PK/PD modeling results further suggest a potential improvement in patient experience and health outcomes. The dosing regimens for the phase 2 study (50 mg LD or 20 mg LD) planned for the treatment of MG are multi-fold lower than chronic regimens evaluated previously in the TOUR006 development program in Crohn's disease (CD) (NCT01345318), indicating desirable clinical response outcomes due to the lower doses being used. For example, 50 mg dose is 2-fold less than and 20 mg dose is 5-fold less than the 100 mg dose evaluated in the Crohn's extension study, respectively. In addition, it is predicted that the treatment duration in MG will be finite and limited (e.g., 6 months) and thus further mitigates risk for adverse effects that are dependent upon exposure duration.

Overall, the PK/PD Modeling predicts that the planned dosing regimens of TOUR006 offer broad, deep and durable effects, an appropriate safety profile and low drug administration burden which supports a patient-centric treatment experience.

INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method of treating myasthenia gravis (MG) comprising administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10.

2. The method of claim 1, wherein the anti-IL-6 antibody or antibody fragment comprises a heavy chain polypeptide comprising a polypeptide having at least 98% identity to SEQ ID NO: 1 and a light chain polypeptide comprising a polypeptide having at least 98% identity to SEQ ID NO: 7.

3. The method of claim 1, wherein the anti-IL-6 antibody or antibody fragment comprises a heavy chain polypeptide having the sequence of SEQ ID NO: 1 and a light chain polypeptide having the sequence of SEQ ID NO: 7.

4. The method of any one of claims 1-3, wherein the anti-IL-6 antibody or antibody fragment containing said CDRs is contained in a pharmaceutical composition that comprises said anti-IL6 antibody or antibody fragment and a pharmaceutically acceptable carrier.

5. The method of claim 1, wherein said therapeutically effective dose is between 5 mg to 100 mg.

6. The method of claim 1, wherein said therapeutically effective dose is administered subcutaneously.

7. The method of claim 1, wherein said therapeutically effective dose is administered from every week to every 24 weeks.

8. The method of claim 7, wherein said therapeutically effective dose is administered every 4 weeks.

9. The method of claim 1, further comprising:
(a) administering a loading dose of the anti-IL-6 antibody or antibody fragment to the patient for at least the first dose during a loading regimen; and
(b) thereafter administering a maintenance dose of the anti-IL-6 antibody or antibody fragment to the patient during a maintenance regimen.

10. The method of claim 9, wherein the loading regimen comprises administering the loading dose every 1 week, every 2 weeks, or every 4 weeks.

11. The method of claim 9, wherein the maintenance regimen comprises administering the maintenance dose every 4 weeks, every 8 weeks, every 12 weeks, or every 24 weeks.

12. The method of claim 9, wherein the loading dose is greater than or equal to the maintenance dose.

13. The method of claim 9, wherein the loading dose is less than the maintenance dose.

14. The method of claim 9, wherein the loading regimen comprises one loading dose of 50 mg; and wherein the maintenance regimen comprises the maintenance dose of 20 mg every 4 weeks for a total of 24 weeks.

15. The method of claim 9, wherein the loading regimen comprises one loading dose of 20 mg; and wherein the maintenance regimen comprises the maintenance dose of 10 mg every 4 weeks for a total of 24 weeks.

16. The method of claim 1, wherein the patient is positive for anti-Acetylcholine Receptor (AChR), or anti-muscle specific kinase (MuSK) antibody.

17. The method of claim 1, wherein the patient has seronegative MG.

18. The method of claim 1, wherein the patient has elevated high-sensitivity C-reactive protein (hsCRP) of at least 2 mg/L, 5 mg/L, or 10 mg/L.

19. The method of claim 1, wherein the patient has elevated serum IL-6.

20-40. (canceled)

41. The method of claim 1, wherein said method of treatment achieves one or more of the following results:
(a) a reduction in serum concentrations of anti-AChR antibody from baseline by at least 40%, 45%, or 50%;
(b) a reduction in MG-ADL score from baseline by at least 2, 2.5, 3, 3.5, 4, 4.5 or 5 points;
(c) a reduction in QMG score from baseline by at least 3, 3.5, 4, 4.5, or 5 points;
(d) a reduction in Myasthenia Gravis Composite (MGC) score from baseline by at least 3, 3.5, 4, 4.5, or 5 points; or
(e) a reduction in Myasthenia Gravis Quality of Life 15 Scale (MG-QOL 15r) score from baseline by at least 4, 5, 6, 7, 8, or 9 points.

42-52. (canceled)


SEQUENCE LISTING (12 sequences)

SEQ ID NO: 1 - protein, 441 aa, Homo sapiens
QVQLQQWGAGLLKPSETLSLTCAIYGGSFREYYWSWIRQPPGKGLEWIGEIFHSGSTNYNPSLKSRVTISVDTSKNQFSLKLSSVTAADTAVYYCAREELDDFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 5 aa, Homo sapiens
EYYWS

SEQ ID NO: 3 - protein, 16 aa, Homo sapiens
EIFHSGSTNYNPSLKS

SEQ ID NO: 4 - protein, 8 aa, Homo sapiens
EELDDFDI

SEQ ID NO: 5 - protein, 116 aa, Homo sapiens
QVQLQQWGAGLLKPSETLSLTCAIYGGSFREYYWSWIRQPPGKGLEWIGEIFHSGSTNYNPSLKSRVTISVDTSKNQFSLKLSSVTAADTAVYYCAREELDDFDIWGQGTMVTVSS

SEQ ID NO: 6 - DNA, 1326 bp, Homo sapiens
  source 1..1326: mol_type = other DNA
caggtgcagctacagcagtggggcgcaggactgttgaagccttcggagaccctgtccctcacctgcgctatctatggtgggtccttcagggagtactactggagctggatccgccagcccccagggaaggggctggagtggattggggaaatctttcatagtggaagcaccaactacaacccgtccctcaagagtcgagtcaccatatcagtagacacgtccaagaaccagttctccctgaagctgagctctgtgaccgccgcggacacggctgtgtattactgtgcgagagaggaattagatgattttgatatctggggccaagggacaatggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtagtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - protein, 213 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYKSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 8 - protein, 11 aa, Homo sapiens
RASQGISSWLA

SEQ ID NO: 9 - protein, 7 aa, Homo sapiens
AASSLQS

SEQ ID NO: 10 - protein, 9 aa, Homo sapiens
QQYKSYPRT

SEQ ID NO: 11 - protein, 107 aa, Homo sapiens [sequence omitted]

SEQ ID NO: 12 - DNA, 642 bp, Homo sapiens
  source 1..642: mol_type = other DNA
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagtagcctgcagcctgaagattttgcaacttattactgccaacagtataaaagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt